(19) United States
(12) Patent Application Publication
(10) Pub. No.: US 2024/0373829 A1
(43) Pub. Date: Nov. 14, 2024

(54) GENETIC RESISTANCE TO VIRAL DISEASE IN SALMONID FISH

(71) Applicant: THE UNIVERSITY COURT OF THE UNIVERSITY OF EDINBURGH, Edinburgh (GB)

(72) Inventors: Ross HOUSTON, Midlothian (GB); Jonathan PAVELIN, Edinburgh (GB); Diego Robledo SANCHEZ, Midlothian (GB); Yehwa JIN, Midlothian (GB)

(21) Appl. No.: 18/690,014

(22) PCT Filed: Sep. 5, 2022
(86) PCT No.: PCT/GB2022/052254

§ 371 Date: Mar. 7, 2024

(30) Foreign Application Priority Data
Sep. 8, 2021 (GB) 2112807.9

(51) Int. Cl. A01K 67/0275; A61K 31/708; C07K 14/46; C12N 15/90; C12Q 1/6876

(52) U.S. Cl. CPC A01K 67/0275; A61K 31/708; C07K 14/461; C12N 15/90; C12Q 1/6876; A01K 2227/40; A01K 2267/02; C12Q 2600/124; C12Q 2600/156; C12Q 2600/158

(57) ABSTRACT

The present disclosure relates to methods of screening salmonids for increased resistance to viral infection, such as infectious pancreatic necrosis virus (IP-NV) infection. The present disclosure also relates to fish which have been genetic modified to have increased resistance to viral/IPNV infection. The present disclosure further relates to the use of these fish, which have been identified. or genetically modified to have increased genetic resistance. in aquaculture breeding programs and/or production. The present disclosure further relates to the use of small molecules which target NAE1 and their use in therapy or prevention of viral/IPNV infection.

### FIELD OF THE DISCLOSURE

The present disclosure relates to methods of screening salmonids for increased resistance to viral infection, such as infectious pancreatic necrosis virus (IPNV) infection. The present disclosure also relates to fish which have been genetic modified to have increased resistance to viral/IPNV infection. The present disclosure further relates to the use of these fish, which have been identified, or genetically modified to have increased genetic resistance, in aquaculture breeding programs and/or production. The present disclosure further relates to the use of small molecules which target NAE1 and their use in therapy or prevention of viral/IPNV infection.

### BACKGROUND TO THE DISCLOSURE

Understanding the genetic regulation of traits of importance to farmed animal production is key to guiding optimal use of genomic information in selective breeding programmes¹. Such production traits are typically underpinned by a polygenic architecture, with many loci of minor effect contributing to their heritability¹. However, there are exceptions where major effect loci segregate within farmed animal populations, and a single genomic region underlies the majority of genetic variation in a trait of interest. One such example is the case of host resistance to infectious pancreatic necrosis virus (IPNV) in Salmonids, such as Atlantic salmon, a species with a global aquaculture production of >2.4 million tonnes, worth >$17.1 billion USD in 2018. A major quantitative trait locus (QTL) affecting resistance was described by two independent groups^{2,3} and explains 80-100% of the genetic variance in mortality due to the disease. The application of marker-assisted selection for the identified resistance allele has exemplified the benefits to be gained from applied molecular genetics, contributing to a reduction in IPN mortalities from tens-of-millions in 2009 down to negligible levels five years later. However, while the epithelial cadherin gene (cdh1) has been previously suggested to play a role in mediating the QTL effect⁵, there are still significant knowledge gaps in the underlying functional mechanisms underlying the QTL.

IPNV is the prototypical birnavirus (genus Aquabirnaviridae, family Birnaviridae), and consists of an unenveloped capsid containing a bisegmented double-strand RNA genome. IPNV is capable of causing high levels of morbidity and mortality in farmed salmonid species, including Atlantic salmon (Salmo salar) and rainbow trout (Oncorhynchus mykiss). Clinical signs of IPNV include pancreatic necrosis accompanied by abdominal swelling, darkening of the skin and erratic swimming behaviour. IPNV outbreaks typically occur at two distinct points of the salmon aquaculture production cycle; in first feeding fry in freshwater and in smolts after transfer to seawater⁵. Protection during freshwater production can be partially achieved through vigilant monitoring and biosecurity, but this is ineffective in open seawater pens due to constant exposure to the ocean environment. Vaccination is also partially effective, but generally only feasible for helping prevent disease in the later lifecycle post-smolt stage of production.

A large and significant genetic component to IPN resistance at both crucial stages of the Atlantic salmon lifecycle has been demonstrated, and the major QTL explaining most of this genetic variation has been well described in both Scottish and Norwegian populations, with evidence for at least partial dominance of the resistance-associated allele^{2,3}. The cdh1 gene was suggested to play a functional role in host resistance to IPNV via prevention of entry of the virus into cells⁴. However, the purported functional mutation in this gene was only in partial linkage disequilibrium with the inferred QTL genotype (r²˜0.58) meaning that significant other factors must contribute to the QTL effect. Furthermore, the proposed mechanism of an amino acid change in Cdh1 preventing viral entry to the cells is unlikely since IPNV can successfully replicate in fully resistant salmon fry⁶. Furthermore, the mechanism of viral entry into cells has now been demonstrated to be micropinocytosis, which is inconsistent with the proposed clathrin-mediated endocytosis associated with cdh1⁵.

### SUMMARY OF THE DISCLOSURE

The present disclosure is based on a series of genetics, genomics, genome editing, and functional virology experiments, which were performed to investigate the putative function of candidate genes within the major QTL region in Atlantic salmon. Pooled whole genome sequencing of RR (homozygous resistant) and SS (homozygous susceptible) salmon fry was used to discover and functionally annotate all polymorphisms within the QTL region. Host transcriptomic and viral load analysis were performed on RR and SS fry from two families, based on whole fry samples collected at selected timepoints pre-and post-IPNV challenge. Fine mapping using the pooled whole genome sequencing highlighted a number of highly significant SNPs in the QTL region, which clustered around the coding region and putative regulatory regions of the gene NEDD-8 activating enzyme 1 (nae1). Furthermore, nae1 was one of the most significantly differentially expressed genes between RR and SS fry genome-wide, showing notably higher expression levels in resistant fish both constitutively and post-challenge. Following these findings, a series of experiments to disrupt the activity of nae 1 and cdh1 were performed in Atlantic salmon cell lines, using CRISPR-Cas9 knockout and specific molecular inhibitors. The results point to a major role for nae1 but not cdh1 in viral replication in salmon cells, lending significant support to the hypothesis that nae1 is a functional gene mediating the large effect of the QTL on resistance to the virus. Additional studies were conducted on rainbow trout cell lines, which further support the role of nae1 on resistance to virus infection in salmonid fish.

In a first aspect, there is provided a method of identifying whether or not a salmonid fish may display increased resistance to infection by a virus, the method comprising detecting an nucleotide alteration, expression or activity level of the nae1 gene and/or protein in the salmonid fish and determining whether or not the salmonid fish is resistant, or likely to display increased resistance to infection, or likely to have offspring which display increased resistance to infection by the virus, based on the nucleotide alteration, expression or activity level detected.

In accordance with the teaching herein, an altered (i.e. mutant) nucleotide sequence, or level of nae1 gene and/or protein expression or activity, is expected to be associated with increased resistance to virus infection. Correspondingly, a wild-type sequence or normal level of nae1 gene and/or protein expression or activity, is expected to be associated with a reduced resistance to virus infection. Thus, in one embodiment, the methods of identifying whether or not a salmonid fish may display increased resistance to infection by a virus are intended to identify fish which display a reduced level of expression or activity of the nae1 gene and/or protein, in order to identify fish which are likely to display an increased resistance to infection by a virus. In order to ascertain whether or not there has been an nucleotide alteration, or change in expression or activity of the nae1 and/or protein, it may be appropriate to carry out a comparison with a wild-type nae1 gene, in order to determine if there is a nucleotide alteration and/or a change in the expression level or activity of the nae1 gene or protein.

NAE1: The protein encoded by nae1 binds to the beta-amyloid precursor protein in humans. Beta-amyloid precursor protein is a cell surface protein with signal-transducing properties, and it is thought to play a role in the pathogenesis of Alzheimer's disease. In addition, the encoded protein can form a heterodimer with UBE1C and bind and activate NEDD8, a ubiquitin-like protein. This protein is required for cell cycle progression through the S/M checkpoint. Three transcript variants encoding different isoforms have been found for this gene. The nae1 gene has been identified in many organisms, including fish species. In sockeye salmon, for example, it has been identified in chromosome 28.

In one embodiment, the virus to which salmonid fish is resistant is a birnavirus. Birnaviruses are double-stranded RNA viruses, which infect Salmonids. In one embodiment, the birnavirus is infectious pancreatic necrosis virus (IPNV). The disease mainly affects young salmonids, such as trout and salmon, of less than six months old, although adult fish may carry the virus without showing symptoms. Thus, detection of the virus in young (<6 months old) and/or older fish, is of relevance to this disclosure.

In the context of the present disclosure, salmonid fish include all fish in the Salmonidae family. Salmonids are coldwater fishes and include salmon (such as Atlantic, Sockeye, Steelhead, Coho and Chinook salmon), trout (such as rainbow and brown trout), chars, freshwater whitefishes, and graylings. In one embodiment, the salmonid fish is a trout, such as rainbow trout.

In accordance with this disclosure, resistance to infection may be, in one teaching, correlated in terms of survival during an infection and, in another teaching, an increase in survival time during an infection. In one teaching both an increase in survival and an increase in survival time (days to death) may be taken into account.

A fish that is determined to have increased resistance to virus infection according to this disclosure is more likely than normal to produce offspring that have a higher than normal chance of having increased resistance to viral infection. Consequently, in a further aspect of the disclosure, there is provided a method of selecting a salmonid fish for use as broodstock, wherein the salmonid fish is selected. based on a method as described herein, to have increased resistance to viral infection. Either or both male and female fish which are identified as having increased resistance to virus infection may be selected for use as broodstock.

Conversely, a salmonid fish predicted by the methods as described herein, as not having increased resistance to viral infection, would not be selected as broodstock.

In accordance with the above and the teaching herein, in a further aspect there is provided a population of salmonids, which have been obtained from at least one male and at least one female salmonid, which has been identified by a method as described herein to have increased resistance to virus infection, or which have been genetically modified in accordance with the teaching herein, to have increased resistance to virus infection.

In a further embodiment, the teaching of the present disclosure may be used in Marker Assisted Selection (MAS), wherein salmonid fish, which are enrolled in a breeding program are checked in accordance with a method as described hereinabove, for their expression level of the nae1 gene and/or protein. This could take the form of a diagnostic genetic test to identify one or more nucleotide alterations in the nae1 gene or gene locus and/or studying expression levels and/or activity of NAE1. For example, salmonid fish having one or more nucleotide alterations as identified herein as increasing resistance to virus infection, may be placed into a breeding program in order to select for offspring that also carry such nucleotide alterations. Accordingly, the nucleotide alterations can be used to non-lethally screen potential broodstock for increased resistance to virus infection. For example, a piece of a fin tissue can be obtained from a fish from a breeding program, and DNA can be extracted and analyzed to determine whether one or more nucleotide alterations in the identified nae1 gene, is present. If the one or more nucleotide alteration/SNPs associated with resistance to virus infection are present, that fish would be desirable to include in a breeding program.

Said nucleotide alteration(s) (or mutations) may be a substitution, deletion, inversion, addition or multiplication (e.g. duplication) of one or more nucleotides, within the nae1 gene sequence, or in a putative regulatory region adjacent to the nae1 gene, which is associated with expression of nae1. This putative regulatory region comprises up to 100, 50, or 25 kb up and downstream of the nae1 gene. In one embodiment, the nucleotide alteration is a SNP, which alters the expression level of nae1. A single-nucleotide polymorphism (SNP) is a substitution of a single nucleotide at a specific position in the genome that is present in a sufficiently large fraction of the population (e.g. 1% or more). Typically, although not exclusively and without wishing to be bound by theory, the nucleotide alteration/SNP may result in a difference in RNA and/or protein expression levels of the nae1 gene or may result in alternative splicing and resulting expression of nae1. The nucleotide alteration may also result in a difference in protein amino acid sequence and/or protein structure, affecting NAE1 activity.

Exemplary SNPs from the relevant locus on chromosome 26 of the Atlantic salmon genome include:

| Position | Allele |
|---|---|
| 14187161 | T/G |
| 14885284 | T/C |
| 14967309 | C/G |
| 15004590 | T/G |
| 15014829 | T/C |
| 15017459 | T/A |
| 15026219 | A/G |
| 15039085 | A/C |
| 15053849 | G/A |
| 15054366 | A/G |
| 15059304 | G/A |
| 15102250 | G/C |
| 15109977 | A/C |
| 15133823 | G/A |
| 15192533 | T/C |
| 15216801 | A/— |
| 15218171 | AT/— |
| 15927162 | A/C |
| 16401284 | T/G |
| 16943333 | G/G |
| 17373181 | T/G |

(Numbering according to the NCBI database, Atlantic salmon genome assembly GCA_000233375.4)

In one embodiment the method comprises identifying if said one or more nucleotide alterations (or mutations) occur on both copies of the chromosomes carrying the nae1 gene and is considered homozygous for the alteration, or occurs on only one chromosome and is therefore considered as being heterozygous for the alteration. In one embodiment, the method identifies one or more homozygous nucleotide alterations.

A person skilled in the art will appreciate that a number of methods can be used to determine the presence of the genetic alterations/SNPs identified in the present disclosure. For example a variety of techniques are known in the art for detecting a gene alteration/SNP within a sample, including genotyping, microarrays (also known as SNP arrays, or SNP chips), Restriction Fragment Length Polymorphism, Southern Blots, SSCP, dHPLC, single nucleotide primer extension, allele-specific hybridization, allele-specific primer extension, oligonucleotide ligation assay, and invasive signal amplification, Matrix-assisted laser desorption/ionization time-of-flight (MALDI-TOF) mass spectrometry, and Fluorescence polarization (FP).

Accordingly, the nucleotide alterations/SNPs may be detected by genotyping. Methods of genotyping are well known in the art. In one method, primers flanking the nucleotide alteration/SNP are selected and used to amplify the region comprising the SNP. The amplified region is then sequenced using DNA sequencing techniques known in the art and analyzed for the presence of the nucleotide alteration/SNP.

In another embodiment, the method of determining a nucleotide alteration/SNP comprises using a probe. For example, in one embodiment an amplified region comprising the nucleotide alteration/SNP is hybridized using a composition comprising a probe specific for the nucleotide alteration/SNP under stringent hybridization conditions.

Thus, the disclosure further teaches isolated nucleic acids that bind to nucleotide alterations/SNPs at high stringency that are used as probes to determine the presence of the gene alteration/SNP. In a particular embodiment, the nucleic acids are labeled with a detectable marker. The marker or label is typically capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The term “probe” refers to a nucleic acid sequence that will hybridize to a nucleic acid target sequence. In one example, the probe hybridises to a sequence comprising a specific nucleotide alteration/SNP or its complement, under stringent conditions, but will not to the corresponding wild-type allele or its complement. The length of probe depends on the hybridization conditions and the sequences of the probe and nucleic acid target sequence. In one embodiment, the probe is an oligonucleotide of 8-50 nucleotides in length, such as, 8-10, 8-15, 11-15, 11-20, 16-20, 16-25, 21-25, or 15-40 nucleotides in length.

In a further embodiment, there is provided a kit for use in one or more of the identification methods described herein, the kit comprising one or more probes for hybridising to said one or more nucleotide alterations within the nae1 gene and/or regulatory region associated with expression of nae1. In one embodiment, the kit only comprises probes for hybridising to said one or more nucleotide alterations within the nae1 gene. That is the kits does not comprise probes capable of specifically hybridizing under stringent conditions to any other genes or within the chromosome(s) in which the nae1 gene is located, other than one or more probes, which may be used for positive control purposes. The probes in the kit may comprise or consist of 1, 2, 3, 4, 5, 10, 15, 20, 25, 30, 40, 50, 75, 100, 500, or 1000 probes, which are designed to specifically hybridise to said one or more nucleotide alterations within the nae1 gene, as identified herein.

A kit could take any variety of forms. In one embodiment the kit may comprise a substrate upon which said probe(s) are bound or otherwise attached to. The probes may be provided in a form of array, where individual probes of bound/adhered to specific and discernable locations on the substrate, so as to easily facilitate with identifying which probes bind to test nucleic acid.

The skilled addressee is well aware of other components such as reagents, buffers, nucleotides etc, which may be included in a kit.

By “stringent conditions” it is meant that conditions are selected which promote selective hybridization between two complementary nucleic acid molecules in solution. Hybridization may occur to all or a portion of a nucleic acid sequence molecule. Those skilled in the art will recognize that the stability of a nucleic acid duplex, or hybrids, is determined by the Tm, which in sodium containing buffers is a function of the sodium ion concentration and temperature (Tm=81.5×−16.6 (Log10[Na+])+0.41 (% (G+C)−600/l), or similar equation).

Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm. for example if nucleic acid molecules are sought that have a >95% identity, the final wash temperature will be reduced by about 5° C. Based on these considerations those skilled in the art will be able to readily select appropriate hybridization conditions. In preferred embodiments. stringent hybridization conditions are selected. By way of example the following conditions may be employed to achieve stringent hybridization: hybridization at 5x sodium chloride/sodium citrate (SSC)/5× Denhardt's solution/1.0% SDS at Tm −5°° C. for 15 minutes based on the above equation, followed by a wash of 0.2× SSC/0.1% SDS at 60° C. It is understood, however, that equivalent stringencies may be achieved using alternative buffers, salts and temperatures. Additional guidance regarding hybridization conditions may be found in: Current Protocols in Molecular Biology, John Wiley & Sons, N. Y., 1989, 6.3.1-6.3.6 and in: Sambrook et al., Molecular Cloning, a Laboratory Manual, Cold Spring Harbor Laboratory Press, 1989, Vol.3. Nucleic acid sequences that are primers are useful to amplify DNA or RNA sequences containing a nucleotide alteration/SNP of the present disclosure. Accordingly, in one teaching, the disclosure provides a composition comprising at least one isolated nucleic acid sequence that is a specific probe or primer able to hybridise and/or amplify a sequence comprising a nucleotide alteration/SNP identified in nae1 or the regulatory region of nae1. A person skilled in the art would understand how to identify and test probes/primers that are useful for detecting/amplifying sequences containing nucleotide alterations/SNPs identified within the nae1 gene or its regulatory region.

In a further embodiment, nucleotide alteration(s)/SNPs may detected using a primer extension assay. Briefly, an interrogation primer is hybridised to a sequence of nucleotides immediately upstream of the nucleotide alteration/SNP nucleotide. A DNA polymerase then extends the hybridized interrogation primer by adding a base that is complementary to the nucleotide alteration/SNP. The primer sequence containing the incorporated base is then detected using methods known in the art. In one embodiment, the added base is a fluorescently labeled nucleotide. In another embodiment, the added base is a hapten-labelled nucleotide recognized by antibodies.

Such detection techniques known in the art include microarrays, hybridization assays, molecular beacons, Dynamic allele-specific hybridization (DASH) and/or combinations of these.

The nucleotide alterations/SNPs described herein are optionally detected using restriction enzymes. For example, amplified products can be digested with a restriction enzyme that specifically recognizes a sequence comprising one of the nucleotide alteration/SNP alleles, but does not recognize a wild-type allele (or vice versa). In one embodiment PCR is used to amplify DNA comprising a nucleotide alteration/SNP, amplified PCR products are subjected to restriction enzyme digestion under suitable conditions and restriction products are assessed. If for example a specific nucleotide alteration/SNP allele corresponds to a sequence digested by the restriction enzyme, digestion is indicative of detecting that particular nucleotide alteration/SNP allele. Restriction products may be assayed electrophoretically as is common is the art.

Nucleotide alteration/SNP alleles can also be detected by a variety of other methods known in the art. For example, PCR and RT-PCR and primers flanking the nucleotide alteration/SNP can be employed to amplify sequences and transcripts respectively in a sample comprising DNA (for PCR) or RNA (for RT-PCR). The amplified products are optionally sequenced to determine which of the nucleotide alteration/SNP alleles is present in the sample.

In one embodiment, the disclosure includes isolated nucleic acid molecules that selectively hybridize under stringent conditions to nae1 comprising one or more alterations/mutations. A further embodiment includes an isolated nucleic acid molecule that selectively hybridizes to a nucleic acid comprising an altered allele or its complement. The phrase “specifically hybridizes to an altered allele or its complement” means that under the same conditions, the isolated nucleic acid sequence will preferentially hybridize to one of the altered alleles or its complement, as compared to a wild-type allele. The term “hybridize” refers to the sequence specific non-covalent binding interaction with a complementary nucleic acid. In a preferred embodiment, the hybridization is under high stringency conditions.

In one embodiment, the present disclosure is directed to identifying an expression level of nae1 or NAE1 in a salmonid fish. in order to ascertain whether or not the expression level is increased or decreased with respect to a wild-type salmonid fish.

Various techniques for determining the expression level nae1 are known to the skilled addressee, including, but not limited to, Q-PCR, RT-PCR, microarray, high-throughput sequencing, continuous analysis of expression (SAGE) and digital gene expression (DGE). In certain embodiments, the expression level of nae1 can be determined with respect to various characteristics of the expression product of the gene, such as exons, introns, protein epitopes and protein activity.

The expression product to be assayed can be, for example, RNA or a polypeptide. The expression product may be fragmented. For example, the assay can use primers complementary to the target sequence of the expression product, so that a complete transcript, as well as a fragmented expression product containing the target sequence, can be measured.

RNA expression products can be assayed directly or by detection of cDNA obtained from PCR-based amplification methods such as quantitative reverse transcription polymerase chain reaction (qRT-PCR) (eg, U.S. Pat. No. 7,587,279). Polypeptide expression products may be assayed using immunohistochemistry (IHC) by proteomic techniques, or functional assays, which are designed to detect a level of protein activity. In addition, microarrays may be used to assay both RNA and polypeptide expression products.

Gene expression profiling methods include methods based on polynucleotide hybridization analysis, methods based on polynucleotide sequencing, and methods based on proteomics. Exemplary methods for quantifying the expression of RNA in a sample include Northern blotting and in situ hybridization known in the art (Parker & Barnes, Methods in Molecular Biology 106:247-283 (1999)), Ribonuclease (RNAse) protection assay (Hod, Biotechniques 13:852-854 (1992)), and PCR-based methods such as reverse transcription PCR (RT-PCR) (Weis et al., Trends in Genetics 8:263-H.264 (1992).

NAE1 expression levels may be determined by use of gel electrophoresis, immunoassay (such as ELISA), Western blotting and spectrophotometric techniques known in the art, for example. NAE1 activity levels may be determined by use of functional assays, such as the detection of downstream gene expression markers, such as IRF3 and/or IRF7. Also, NAE1 activity may be determined using, for example, a NEDD8 conjugation initiation kit (available from Bio-Techne Ltd, UK)

In addition to identifying salmonid fish, which have a nucleotide alteration, altered expression or activity level of the nae1 gene and/or protein, the present disclosure further provides a genetically modified salmonid fish, wherein the fish has been genetically modified such that its genome comprises, consists essentially of, or consists of a mutant nae1 gene or allele.

As described above in relation to salmonid fish, which are detected as having a “natural” nucleotide alteration, altered expression and/or activity level of the nae/gene and/or protein, such genetically modified fish may be use in breeding and aquaculture programs, for example.

The genetically modified fish, may be provided through recombinant molecular biology (for example homologous recombination techniques) or genome editing (such as CRISPR) techniques which result in NAE1 inhibition and/or increased resistance to viral infection. It has also been observed that NAE1 may be inhibited using chemicals. Thus, in a further teaching, there is provided a NAE1 inhibitor for use in the treatment or prevention of viral infection, such as IPNV infection in a salmonid fish. Alternatively, there is provided a NAE1 inhibitor for use in the manufacture of a medicament for the treatment or prevention of viral infection, such as IPNV infection in a salmonid fish.

Examples of types of NAE1 inhibitors useful for the invention include, but are not limited to, a peptide, a peptidomimetic, a small molecule, a polynucleotide, or a polypeptide.

NAE1 inhibition, as used herein, refers to reducing one or more of net nae1 gene expression, net NAE1 protein levels, or net NAE1 activity. Inhibition of NAE1 may include at least about a 10% to a 100% reduction in NAE1 activity level in the presence of, or resulting from, a given dose of the NAE1 inhibitor relative to NAE1 activity level in its absence, e.g., a 10%, 20%, 30%, 40%, 50%, 60%, 70%, 80%, 85%, 90%, 95%, or another percent reduction in NAE1 activity from about 10% to about 100%. In some embodiments, a NAE1 inhibitor is administered to a fish to be treated.

Increased resistance to viral infection as used herein, refers to increased survival during an infection and/or an increase in survival time during an infection. This may include at least a 10% increase in survival rate and/or survival time e.g., a 10%, 20%, 30%, 40%, 50%, 60%, 70%, 80%, 85%, 90%, 95%, or greater, for example. Any increase in resistance to viral infection may simply be made in comparison to a salmonid fish comprising a wild-type nae1 gene and/or displaying a normal level/activity of NAE1.

Recombinant techniques may be used in order to knock-out, or reduce expression of nae1. Transgenic and gene targeting techniques are well known to the skilled addressee and have been adopted in fish⁷. Production and development genetically modified fish may be based on embryo manipulation and homologous recombination technology. Through the targeted modification of a fishes genome, modified genetic information is inherited in vivo. Thus, the skilled addressee is aware of, for example. homologous recombination techniques, which can be used in order to cause a knock-out of the nae1 gene. More recent techniques which employ site-specific nucleases, such as zinc finger nucleases, TALENS, or CRISPR techniques are known and are described herein.

In some embodiments, nae1 expression may be inhibited by use of a polynucleotide, which may inhibit nae1 expression or NAE1 activity, by at least one of a number of different mechanisms as described.

### RNA Interference

In some embodiments a polynucleotide nae1 inhibitor acts by reducing expression of NAE1 protein by targeting its mRNA. For example, the polynucleotide can be an RNAi.

The terms “RNA interference”, “RNAi” or “gene silencing” refer generally to a process in which a double-stranded RNA molecule reduces the expression of a nucleic acid sequence with which the double-stranded RNA molecule shares substantial or total homology. However, it has been shown that RNA interference can also be achieved using non-RNA double stranded molecules (see, for example, US 20070004667).

In some embodiments, a NAE1 inhibitor comprises nucleic acid molecules comprising and/or encoding double-stranded regions for RNA interference against the nae1 mRNA encoding NAE1. The nucleic acid molecules are typically RNA but may comprise chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The nucleic acid molecule may of course comprise unrelated sequences which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe nucleic acid molecules that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules can be used to epigenetically silence genes at both the post-transcriptional level or the pre-transcriptional level. In a non-limiting example, epigenetic regulation of gene expression by siRNA molecules can result from siRNA mediated modification of chromatin structure to alter gene expression.

By “shRNA” or “short-hairpin RNA” is meant an RNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to about 15 nucleotides which forms a single-stranded loop above the stem structure created by the two regions of base complementarity.

Included shRNAs are dual or bi-finger and multi-finger hairpin dsRNAs, in which the RNA molecule comprises two or more of such stem-loop structures separated by single-stranded spacer regions.

Once designed, the nucleic acid molecules comprising a double-stranded region can be generated by any method known in the art, for example, by in vitro transcription, recombinantly, or by synthetic means.

Modifications or analogs of nucleotides can be introduced to improve the properties of the nucleic acid molecules. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl-and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

### Polynucleotides Encoding Peptides or Polypeptides

In some embodiments, a polynucleotide-based NAE1 inhibitor encodes a polypeptide, so that delivery of the polynucleotide to fish results in expression of an encoded peptide or polypeptide NAE1 protein inhibitor.

In some embodiments, the polynucleotide NAE1 inhibitor encodes a programmable nuclease which inhibits NAE1 activity by inactivating or reducing expression of the nae 1 gene. As used herein, the term “programmable nuclease” relates to nucleases that are “targeted” (“programmed”) to recognize and edit a pre-determined genomic location. In some embodiments the encoded polypeptide is a programmable nuclease “targeted” or “programmed” to introduce a genetic modification into the nae1 gene or regulatory region thereof. In some embodiments, the genetic modification is a deletion or substitution in the nae1 gene or in a regulatory region thereof.

In some embodiments, the programmable nuclease may be programmed to recognize a genomic location by a combination of DNA-binding zinc-finger protein (ZFP) domains. ZFPs recognize a specific 3-bp in a DNA sequence, a combination of ZFPs can be used to recognize a specific a specific genomic location. In some embodiments, the programmable nuclease (TALEN) may be programmed to recognize a genomic location by transcription activator-like effectors (TALEs) DNA binding domains. In an alternate embodiment, the programmable nuclease may be programmed to recognize a genomic location by one or more RNA sequences. In an alternate embodiment, the programmable nuclease may be programmed by one or more DNA sequences. In an alternate embodiment, the programmable nuclease may be programmed by one or more hybrid DNA/RNA sequences. In an alternate embodiment, the programmable nuclease may be programmed by one or more of an RNA sequence, a DNA sequences and a hybrid DNA/RNA sequence.

Programmable nucleases that can be used in accordance with the present disclosure include, but are not limited to, RNA-guided engineered nuclease (RGEN) derived from the bacterial clustered regularly interspaced short palindromic repeat (CRISPR)-cas (CRISPR-associated) system, zinc-finger nuclease (ZFN), transcription activator-like nuclease (TALEN), and argonautes.

In some embodiments, the nuclease is a RNA-guided engineered nuclease (RGEN). In some embodiments the RGEN is from an archaeal genome or is a recombinant version thereof. In some embodiments the RGEN is from a bacterial genome or is a recombinant version thereof. In some embodiments the RGEN is from a Type I (CRISPR)-cas (CRISPR-associated) system. In some embodiments the RGEN is from a Type II (CRISPR)-cas (CRISPR-associated) system. In some embodiments the RGEN is from a Type III (CRISPR)-cas (CRISPR-associated) system. In some embodiments the nuclease is a class I RGEN. In some embodiments the nuclease is a class II RGEN. In some embodiments the RGEN is a multi-component enzyme. In some embodiments the RGEN is a single component enzyme. In some embodiments the RGEN is CAS3. In some embodiments the RGEN is CAS10. In some embodiments the RGEN is CAS9. In some embodiments the RGEN is Cpf1. In some embodiments the RGEN is targeted by a single RNA or DNA. In some embodiments the RGEN is targeted by more than one RNA and/or DNA. In some embodiments the programmable nuclease may be a DNA programmed argonaute (see WO 14/189628).

In some embodiments the polynucleotide NAE1 inhibitor is provided in an expression vector to be delivered in vivo or in vitro to fish cells using any of a number of transfection methods known in the art, e.g., recombinant virus transduction, liposome-based transfection, electroporation, or nano-particle based transfection.

As used herein, an “expression vector” is a DNA or RNA vector that is capable of effecting expression of one or more polynucleotides in a host cell (e.g., a fish embryo). The vector is typically a plasmid or recombinant virus. Any suitable expression vector can be used, examples of which include, but are not limited to, a plasmid or viral vector. In some embodiments, the viral vector is a retrovirus, a lentivirus, an adenovirus, a herpes virus, or an adeno-associated viral vector.

Such vectors will include one or more promoters for expressing the polynucleotide such as a dsRNA for gene silencing. Suitable promoters include, but are not limited to, the retroviral LTR; the SV40 promoter; and the human cytomegalovirus (CMV) promoter. Cellular promoters such as eukaryotic cellular promoters including, but not limited to, the histone, RNA polymerase III (in the case of shRNA or miRNA expression), and β-actin promoters, can also be used.

The term “nucleic acid sequence” (or nucleic acid molecule) refers to a DNA or RNA molecule in single or double stranded form, particularly a DNA encoding a protein or protein fragment according to the invention.

The term “gene” means a DNA sequence comprising a region (transcribed region), which is transcribed into an RNA molecule (e.g. a pre-mRNA, comprising intron sequences, which is then spliced into a mature mRNA) in a cell, operable linked to regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non translated sequence comprising e.g. transcription termination sites.

The terms “protein” or “polypeptide” are used interchangeably and refer to molecules consisting of a chain of amino acids, without reference to a specific mode of action, size, 3-dimensional structure or origin.

As used herein, the term “allele(s)” means any of one or more alternative forms of a gene at a particular locus. In a diploid (or amphidiploid) cell of an organism, alleles of a given gene are located at a specific location or locus (loci plural) on a chromosome. One allele is present on each chromosome of the pair of homologous chromosomes. As used herein, the term “homologous chromosomes” means chromosomes that contain information for the same biological features and contain the same genes at the same loci but possibly different alleles of those genes. Homologous chromosomes are chromosomes that pair during meiosis. “Non-homologous chromosomes”, representing all the biological features of an organism, form a set, and the number of sets in a cell is called ploidy. Diploid organisms contain two sets of non-homologous chromosomes, wherein each homologous chromosome is inherited from a different parent.

As used herein, the term “heterozygous” means a genetic condition existing when two different alleles reside at a specific locus, but are positioned individually on corresponding pairs of homologous chromosomes in the cell. Conversely, as used herein, the term “homozygous” means a genetic condition existing when two identical alleles reside at a specific locus, but are positioned individually on corresponding pairs of homologous chromosomes in the cell. As used herein, the term “locus” (loci plural) means a specific place or places or a site on a chromosome where for example a gene or genetic marker is found. For example, the “nae1 locus” refers to the position on a chromosome where the nae 1 gene (and two nae1 alleles) may be found.

The term “nucleotide alteration”, “mutant” or “mutation” refers to e.g. a fish or gene that is different from the so-called “wild type” variant (also written “wildtype” or “wild-type”), which refers to a typical form of e.g. a fish or gene as it most commonly occurs in nature. A “wild type fish” refers to a fish with the most common phenotype of such fish in the natural population. A “wild type allele” refers to an allele of a gene required to produce the wild-type phenotype. A mutant plant or allele can occur in the natural population or be produced by human intervention, e.g. by mutagenesis, and a “mutant allele” thus refers to an allele of a gene required to produce the mutant phenotype.

As used herein, the term “mutant nae1 allele” refers to a nae1 allele, which directs expression of a significantly reduced amount of functional NAE1 protein than the corresponding wild type allele. This can occur either by the mutant nae1 allele encoding a non-functional NAE1 protein, which, as used herein, refers to a NAE1 protein having no biological activity, a significantly modified and/or a significantly reduced biological activity as compared to the corresponding wild-type functional NAE1 protein, or by the mutant nae1 allele encoding a significantly reduced amount of functional NAE1 protein or no NAE1 protein at all. Such a “mutant nae1 allele” thus comprises one or more mutations in its nucleic acid sequence when compared to the wild type allele, whereby the mutation(s) preferably result in a significantly reduced (absolute or relative) amount of functional NAE1 protein in the cell in vivo.

As used herein, the term “expression level” when applied to a gene or protein is the normalized level of the gene or gene product (e.g., the normalized value determined relative to the DNA or RNA level of the gene or the polypeptide level).

The term “gene product” or “expression product” as used herein refers to RNA transcripts (transcripts) of genes, including mRNA, and polypeptide translation products of such RNA transcripts. The gene product can be, for example, non-spliced RNA, mRNA, splice variant mRNA, microRNA, fragmented RNA, polypeptide, post-translationally modified polypeptide, splice variant polypeptide, and the like.

As used herein, the term “RNA transcript” refers to an RNA transcript of a gene, including, for example, mRNA, non-spliced RNA, splice variant mRNA, microRNA, and fragmented RNA.

As used herein, a “significantly reduced amount of functional NAE1 protein” refers to a reduction in the amount of a functional NAE1 protein produced by the cell comprising a mutant nae1 allele by at least 30%, 40%, 50%, 60%, 70%, 80%, 90%, 95% or 100% (i.e. no functional protein is produced by the cell) as compared to the amount of the functional NAE1 protein produced by the cell not comprising the mutant nae1 allele. In order to determine whether a fish according to the invention, i.e. a fish comprising one or more mutant nae 1 alleles, has a significantly reduced NAE1 level, comparison may be made with a corresponding wild type fish (i.e. of the same genetic background) not comprising said mutant nae1 allele(s) grown under the same conditions.

Whenever reference to a “fish” or “salmonid” according to the invention is made, it is understood that also fish parts (cells. tissues, organs, embryos, sperm, or eggs etc.), progeny of the fish which retain the distinguishing characteristics of the parents (i.e. reduced NAE1 levels), are encompassed herein, unless otherwise indicated.

As used herein, the term “genetically modified” when used in reference to a fish described herein, means a fish with a genome that has been modified by man. A transgenic fish, for example, is a genetically modified fish that contains an exogenous nucleic acid molecule, e.g., a chimeric gene comprising a transcribed region which when transcribed yields a biologically active RNA molecule capable of reducing the expression of an endogenous gene, such as the nae1 gene according to the invention, and, therefore, has been genetically modified by man. In addition, a fish that contains a mutation in an endogenous gene, for example, a mutation in an endogenous nae1 gene, (e.g. in a regulatory element or in the coding sequence) as a result of an exposure to a mutagenic agent, or through genetic modification as described herein, is also considered a genetically modified fish, since it has been genetically modified by man. Furthermore, a fish of a particular species, such as rainbow trout, that contains a mutation in an endogenous gene, for example, in an endogenous nae 1 gene, that in nature does not occur in that particular fish species, as a result of, for example, directed breeding processes, such as marker-assisted breeding and selection, is also considered a genetically modified fish. In contrast, a fish containing only spontaneous or naturally occurring mutations, i.e. a fish that has not been genetically modified by man, is not a “non-genetically modified fish” as defined herein and, therefore, is not encompassed within the invention. One skilled in the art understands that, while a genetically modified fish typically has a nucleotide sequence that is altered as compared to a naturally occurring fish, a genetically modified fish also can be genetically modified by man without altering its nucleotide sequence, for example, by modifying its methylation or glycosylation pattern.

The term “comprising” is to be interpreted as specifying the presence of the stated parts, steps or components, but does not exclude the presence of one or more additional parts, steps or components. A fish comprising a certain trait may thus comprise additional traits. It is understood that when referring to a word in the singular (e.g. fish), the plural is also included herein (e.g. a plurality of fish or fishes). Thus, reference to an element by the indefinite article “a” or “an” does not exclude the possibility that more than one of the element is present, unless the context clearly requires that there be one and only one of the elements. The indefinite article “a” or “an” thus usually means “at least one”.

Unless specifically defined otherwise, all technical and scientific terms used herein shall be taken to have the same meaning as commonly understood by one of ordinary skill in the art (e.g., in cell culture, cell biology, and molecular genetics).

Unless otherwise indicated, the cell culture and molecular biology techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors).

### DETAILED DESCRIPTION

The present disclosure will be further described with reference to the figures, which show:

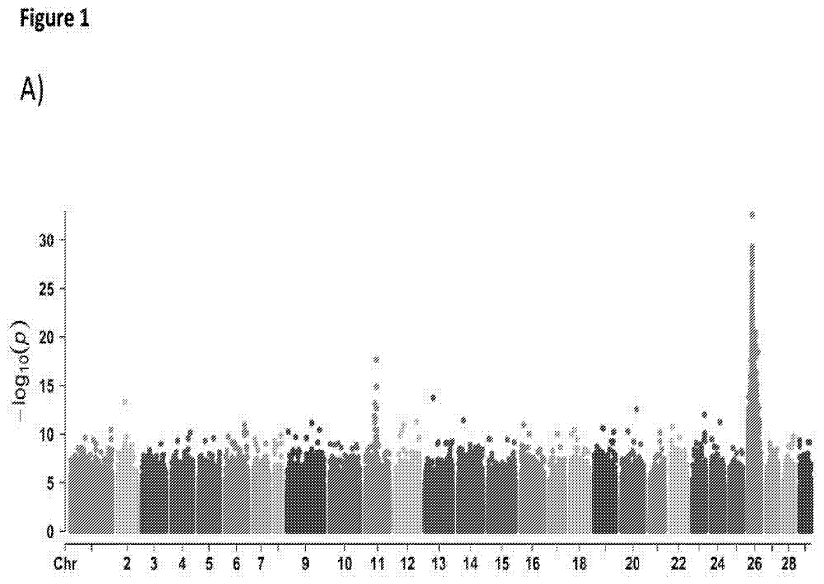

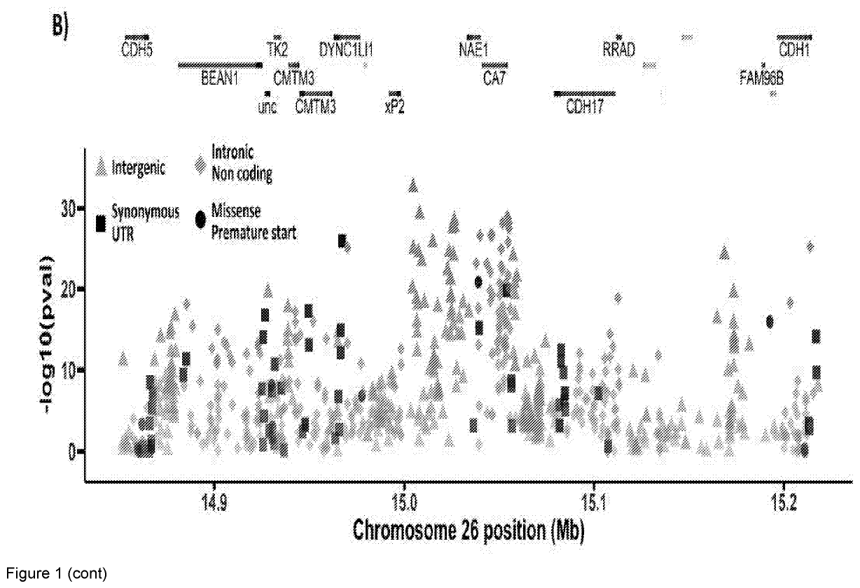

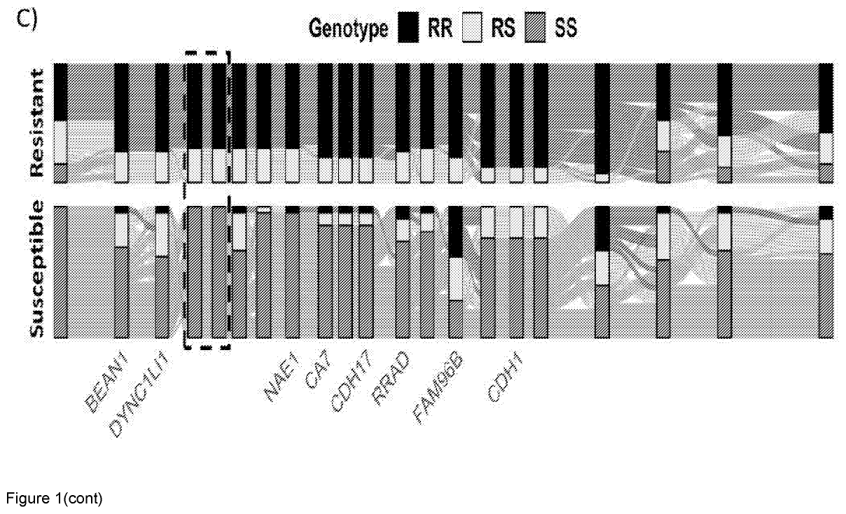

FIG. 1: Genetic mapping and functional characterisation of the IPN resistance QTL region; A) Manhattan plot showing association between genome-wide SNPs and QTL genotype, B) map of annotated genes and functional annotation of SNPs within the most significant QTL region; C) The concordance between significant SNP genotypes and inferred QTL genotypes in offspring from double heterozygous parent families. Each vertical bar represents a SNP in or around the QTL region and each horizontal line represents an individual animal. The boxed area comprises two of the most significant SNPs from the genome-wide scan, and the SNPs that show full concordance between QTL genotype and SNP genotype in susceptible homozygous animals. There are no SNPs with full concordance between QTL genotype and SNP genotype in resistant homozygous animals;

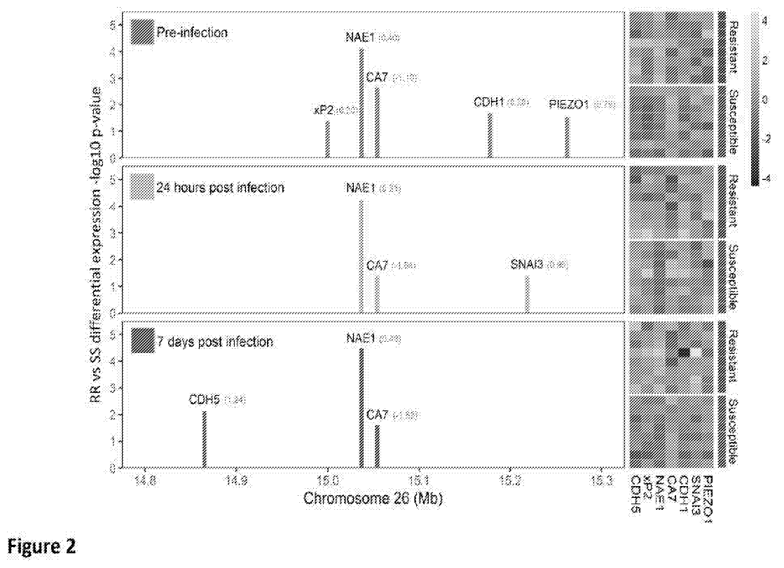

FIG. 2: Differential expression of genes in the IPN resistance QTL region in salmon fry pre-challenge, 1 day post challenge, and 7 days post challenge. The nae1 gene is consistently the most significant differentially expressed gene in the QTL region at all timepoints. The heatmap on the right shows the relative expression levels of the genes in the QTL region showing significant differences between RR and SS fry at any timepoint;

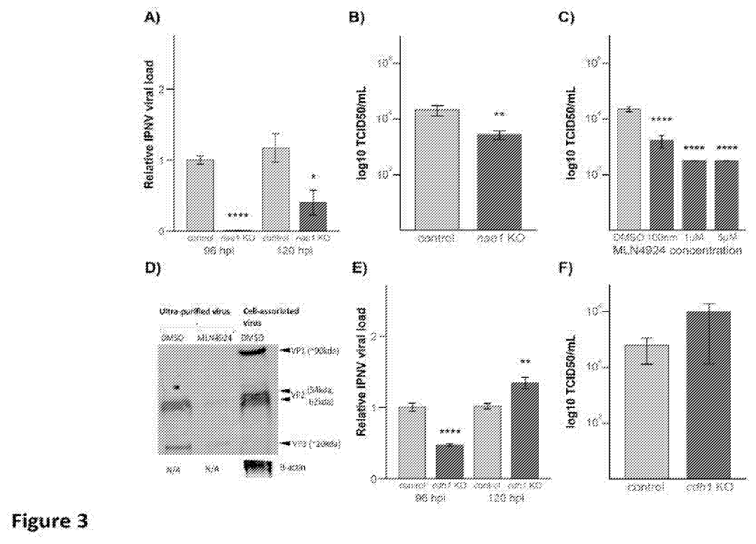

FIG. 3. Assessment of the role of Nae1 and Cdh1 in the replication of IPNV in Atlantic salmon cells. A) IPNV viral load at 96 and 120 hpi in control and nae1 KO SHK-1 infected with IPNV at an MOI of 0.01. Relative expression levels of IPNV VP2 to ef1a in cells were normalised to time-matched control SHK-1 cells. B) Infectivity of IPNV in supernatant at 120 hpi in control and nae1 KO SHK-1 infected with IPNV at an MOI of 0.01 was assessed by TCID50/mL on naïve CHSE-214 cells. C) Infectivity of IPNV in cells and supernatant at 120 hpi in SHK-1 cells treated with 100 nM, 1 UM and 5 UM of MLN4924 or DMSO only and infected with IPNV at an MOI of 0.01 was assessed by TCID50/mL on naïve CHSE-214 cells. D) IPNV viral protein in supernatant of SHK-1 cells treated with 100 nM MLN4924 and infected at an MOI of 0.01 at 120 hpi was analysed by western blotting using an antibody against IPNV viral proteins. E) IPNV viral load at 120 hpi in control and cdh1 KO SHK-1 infected with IPNV at an MOI of 0.01. Relative expression levels of IPNV VP2 to ef1a in cells were normalised to time-matched control SHK-1 cells. F) Infectivity of IPNV in supernatant at 120 hpi in control and cdh1 KO SHK-1 infected with IPNV at an MOI of 0.01 was assessed by TCID50/mL on naïve CHSE-214 cells.

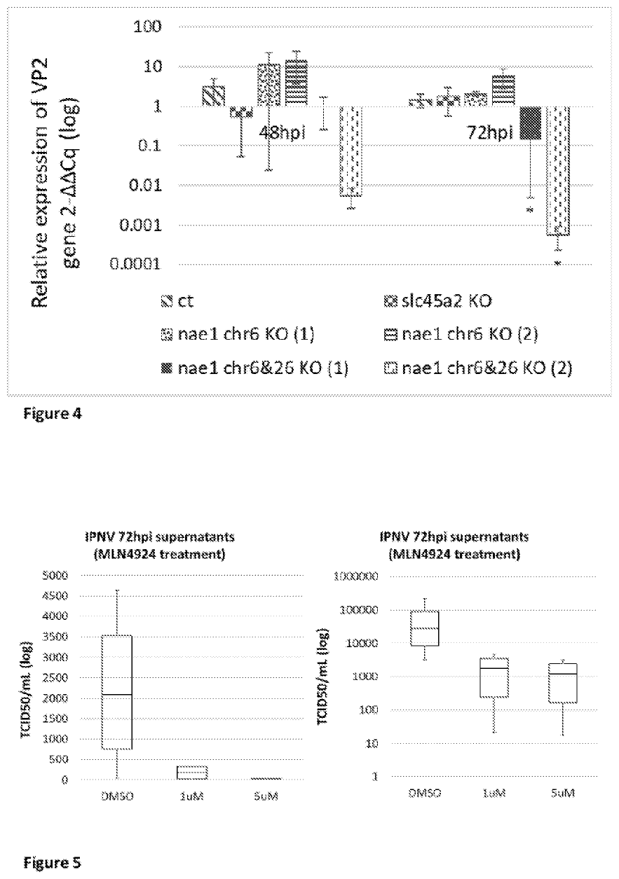

FIG. 4. Changes on the expression patterns of VP2 gene in different cell populations (2nd and 3rd trial, cells were challenged 24 dpe). The graphs show the 2^{-(ΔΔCq)} values estimated from analysis of the RT-qPCR C_{q} data. All cell populations are normalized by wild type electroporated without Cas9/RNA cell population of the corresponding time point.

FIG. 5. Viral output patterns of the three different doses of MLN4924-treated cells, DMSO-only, 1 um and 5 uM at 72 hours post inoculation with the virus.

### MATERIALS AND METHODS

DNA sequencing and fine mapping

23 nuclear families from two yeargroups, derived from a commercial salmon breeding programme (Hendrix Genetics) and where both sire and dam were heterozygous for the IPN resistance QTL, were identified using the methods described in Houston et al.⁸. From each of these families, two fry homozygous for the resistant allele (RR) and two fry homozygous for the susceptibility allele (SS) were identified. Four groups were then established, RR fry from yeargroup 1 (n=22), SS fry from yeargroup 1 (n=22), RR fry from yeargroup 2 (n=24), and SS fry from yeargroup 2 (n=24). Genomic DNA from samples of fry fin tissue taken from individual fry within each group was then pooled at equimolar concentrations, resulting in four pools of genomic DNA. Each of these pools was then sequenced by Edinburgh Genomics (Edinburgh, UK) with 2×125 bp paired-end reads using HiSeq V4 chemistry, aiming for a mean coverage of each pool of 25×. The resulting sequencing reads of the four pools were trimmed from sequencing adapters, then aligned to the Atlantic salmon reference genome (Genbank accession GCA_000233375.4) using bwa-mem (PMID: 20080505). Resulting alignments in bam-format were subjected to duplicate removal using Picard (http://broadinstitute.github.io/picard/) and then variant calling using GATK²⁰ with the Unified Genotyper setting. GATK best practices were used for filtration of variants. Allelic depths observed for each pool at each SNP-position were exported from the vcf-file and were used in analysis to contrast the RR and SS pools within year group by means of determining their absolute differences in allele frequencies.

### Disease Challenge and Gene Expression Analyses

To identify genes which appear to be differentially regulated between IPN resistant and susceptible individuals upon exposure to the virus, challenge experiments for analysis of gene expression patterns were set up as follows. 20 families of Atlantic salmon fry were challenged with IPNV (challenge method described in Houston et al⁸), with two replicate tanks of fry challenged for each family. For each family, the level of mortality was averaged across the two replicate tanks, and mortalities across these families ranged from 0-34% upon challenge termination. Based on the levels of mortality, families J and N were designated susceptible, families Q and T appeared resistant and families I, P, B, O, D, S, C and L were designated as intermediate. To ascertain the QTL genotype of parents of challenged offspring within these families, a fin sample from each parent was removed and genotyped at the IPN QTL-linked microsatellite markers given in Houston et al.⁸ Families B and C were identified as ‘double heterozygote’ families where both parents were putative heterozygotes for the QTL, and, therefore, subsequent gene expression data was considered for these two families only.

### IPNV Testing

Fry mortalities and survivors from the challenged tanks and control tanks were tested for the presence of IPNV using different methods. Fry were weighed, homogenised using sterile pestle, mortar and sand then diluted 1:10 in cell culture medium. The homogenate was centrifuged at 2500×g for 15 min. at 4° C. then the supernatant re-moved and filtered through 0.45 μm filter (Whatman) before inoculation onto 24 h old confluent monolayers of CHSE-214 cells in 96-well cell culture trays for titration. Culture trays were incubated at 15° C. and titres read after 7 days. Wells showing positive cytopathic effect (CPE) for each sample were further tested by ELISA (Test-Line) to confirm the presence of IPNV. Subsequently, for the determination of viral load in the samples used for the microarray experiment, an RT-QPCR assay applied in an accredited commercial laboratory (Integrin Advanced Biosystems, UK) was used.

### Microarray Platform, Hybridization, and Quality Filtering

RNA was extracted, purified, amplified and labelled as described in⁶. The microarray platform and methods for microarray hybridisation are described in⁶. Gene expression patterns between resistant and susceptible offspring within families B and C was analysed as follows. Each family was represented by three tanks each containing 100 fry, one of which was terminated and sampled at 1 day post-challenge (‘time point 1’), one at 7 days post-challenge (‘time point 2’) and one at 20 days post-challenge (‘time point 3’). In addition, a sample of 100 fry from all families was taken prior to challenge (‘time point 0’). To ascertain QTL genotype of sampled individuals at each time point, a fin sample from each offspring was removed and genotyped at the IPN QTL-linked microsatellite markers given in Houston et al.⁸. At each time point, RNA was extracted from six fish of each QTL genotype (i.e. homozygote resistant at the IPN QTL: RR; or homozygote susceptible at the IPN QTL: SS) and hybridised to the Agilent 44K (Atlantic salmon) Oligo Array9. This microarray is comprised of 43,661 probes (partial gene sequences), representing ˜90% of the known Atlantic salmon expressed sequence tags (ESTs)¹³.

Significant differential expression of probes was determined by comparing the mean microarray signal across both time points, using a 3-way ANOVA [factors=QTL genotype (resistant vs. susceptible), family (B or C), and time point (0 or 1)]. To avoid exclusion of genes of potential biological relevance, a nominal threshold of P<0.05 for significance was chosen (i.e. P-values were not corrected for multiple testing).

### Virus and Cell Culture

Salmon head kidney. SHK-1 cells (ATCC 97111106) were propagated at 17.5° C. in L15 media supplemented with 5% FBS, 40 μM-mercaptoethanol, 4 mM glutamine, and Pen Strep antibiotics. Cells were passaged using 0.25% trypsin/EDTA at 80% confluence, pelleted, and split 1:3. Fresh media was added in a 2:1 ratio with conditioned media. Chinook salmon embryo, CHSE-214 cells (ATCC 91041114) were propagated at 17.5° C. in L15 media supplemented with 10% FBS, 4 mM glutamine, and Pen Strep. Cells were passaged using 0.25% trypsin/EDTA at 80% confluence and split 1:6 in fresh media. IPNV VR1318 was provided by Marine Scotland as a crude isolate. Working stocks were established by infecting 80% confluent CHSE-214 cells at a very low MOI in normal cell culture conditions with 2% serum. At approximately 7 dpi, or when >50% of cells exhibited cytopathic effect, supernatant was harvested, debris was pelleted, and the viral stock was aliquoted and frozen at −80° C.

Viral stocks were titrated using plaque assay on CHSE-214. Infections with IPNV were performed on 80% confluent SHK-1 or CHSE-214 cells. Cells were seeded, incubated overnight, washed with PBS prior to overlay with virus diluted in serum free L15. After 2 hours at 15° C., viral inoculum was removed, washed with PBS and the cells were overlaid with 2% FBS media at 15° C.

### Impact of Nae1 and Cdh1 Knockout In Vitro

CRISPR-Cas9 gRNAs were designed for nae1 and cdh1 and selected for maximum on-target efficiency, and minimum off-targets, using the benchling (benchling.com) and the Synthego CRISPR design tools. nae1 KO and cdh1 KO SHK-1 cells were produced by using method described in10. Briefly, SHK-1 cells were transfected with 1 μM Cas9 ribonucleoprotein targeting exon 2 of nae1 or cdh1 (Supplementary Table 1) by electroporation with 2 pulses at 1400V for 20 ms. Genomic DNA was extracted at 7 days post electroporation, the target region was amplified by PCR (Supplementary Table 1), and gene-editing efficiency was assessed by Sanger sequencing and ICE analysis (https://ice.synthego.com), showing 94 and 93% editing efficiency in nae1 KO and cdh1 KO SHK cells, respectively.

Both wild type and KO SHK-1 cells were seeded in 48 well plates and incubated overnight. IPNV was inoculated at MOI of 0.01 in serum free L15 with Pen Strep for 2 hours at 15° C. Then, the viral inoculum was removed and cell monolayers were washed with PBS. 200 UL of L15 with 2% FBS, 40 UM B-mercaptoethanol and Pen Strep was added to each well and incubated at 15° C. At 96 and 120 hpi, supernatants were collected and stored at-70° C. for TCID50 assays. Total RNAs from the cells were extracted using Direct-zol RNA microprep (Zymo Research, Irvine, USA) with DNase I treatment and stored at −70° C. for quantitative real-time PCR (qRT-PCR).

To evaluate the viral load in cells, relative transcript level of IPNV VP2 to ef1a in the total RNAs was analysed by qRT-PCR using Luna Universal One-Step RT-qPCR reagent (NEB, Ipswich, USA) and LightCycler 480 Instrument (Roche, Basel, Switzerland) in duplicates. Each reaction consisted of 0.5 μL RNAs, 1× Reaction Mix, 1× Enzyme Mix, 0.4 μM each primer (Supplementary Table 4) and nuclease-free water up to 10 μL. The thermocycling initiated with reverse transcription at 55° C. for 10 min and initial denaturation at 95° C. for 1 min, followed by 40 cycles of denaturation at 95° C. for 10 sec and extension at 60° C. for 30 sec with plate read, and melt curve analysis. Efficiency and linearity (R²) of each primer pair were checked using serial dilution of total RNAs in duplicates. The relative viral transcript level of IPNV VP2 versus ef1a in the KO SHK-1 cells compared to wild type SHK-1 cells at each timepoint was calculated using 2^{-ΔΔCT}.

TABLE 1
crRNAs and primers for nae1 and cdh1

| | | | | | Amplicon | Annealing |
|---|---|---|---|---|---|---|
| | crRNA | Gene ID | Fw (5′-3′) | Rv (5′-3′) | (bp) | temp (° C.) |
| nae1 | TGATCAATTC | 100194854 | ATGCCAGCCA | TTCCGACGTC | 580 | 69 |
| | CACAGCATC | | ACCAGCATGC | TTCCCCGGAG | | |
| | T | | TT | AC | | |
| cdh1 | TCGGAGTCA | 106587268 | CCACATTTCG | ATTTCCCAGT | 416 | 56 |
| | ACATGTCTA | | CAATCGGGTG | CGGAGCTCGT | | |
| | CC | | AA | TT | | |

TABLE 2
Primers for qRT-PCR

| | | | Amplicon | Eff- | | |
|---|---|---|---|---|---|---|
| | Fw (5′-3′) | Rv (5′-3′) | (bp) | iciency | R2 | Gene ID/ref |
| ef1a | GGCTGGTTCAAG | CAGAGTCACAC | 60 | 1.98 | 0.999 | AF321836 |
| | GGATGGA | CATTGGCG | | | | |
| IPNV | GACCAAGTTCGA | ATCGGCTTGGT | 156 | 1.96 | 0.9997 | FN257531.11 |
| VP2 | CTTCCAGC | GATGTTCTC | | | | |

The infectivity of viral output in the supernatants at 120 hpi was assessed by TCID50 on naïve CHSE-214 cells in 96 well plate format with 4 wells per dilution in 2% serum media. TCID50 was calculated using the Reed and Muench method 12.

To assess the role of cdh1 in IPNV infection, antibody neutralisation was performed using serial 1:1 dilutions of BSA, IPNV-VP2 antibody and cdh1-specific antibody known to recognise Atlantic salmon Cdh14 in a 96 well plate. SHK-1 cells were overlayed with media containing the serially dilute antibody or BSA and incubated at 15° C. for 2 hours and were subsequently infected with IPNV at an MOI of 0.01. At 120 hpi, RNA was harvested from cells and IPNV viral load was assessed by qRT-PCR.

### Impact of Inhibitor of Nae1 Activity (MLN4924) In Vitro

Lyophilised MLN4924 (pevonedistat) was resuspended in DMSO. MLN4924 was titrated for cytotoxicity on CHSE-214 and SHK-1 cells. SHK-1 or CHSE-214 cells were seeded at 80% confluency and treated with 0 (DMSO only), 100 nM, 1 μM or 5 μM MLN4924 for 24 hours prior to inoculation with IPNV at an MOI of 0.01. The impact of the MLN4924 on cell viability was assessed by sampling at 24, 48, 72, and 96 hpi and comparing cell survival in all challenged groups (including the DMSO control) versus the unchallenged control at the same timepoint.

To evaluate the infectivity of viral output, cells and supernatant were harvested at 120 hpi and assessed by TCID50 on naïve CHSE-214 cells. For semi-quantification of viral protein output, western blot against viral proteins was performed. At 120 hpi, supernatant from a 150 mm dishes containing SHK-1 cells treated with either 100 nM MLN4924 or DMSO for 24 hours before infection with IPNV at an MOI of 0.01 was collected, sterile filtered, and ultra-centrifuged at 22000 x g for 1 hour. The ultra-centrifuged virus pellet from the supernatant was resuspended in Laemmli buffer. The cell-associated virus was also analysed by harvesting cells in Laemmli buffer. These samples were separated by PAGE (4-15% Mini-Protean, BIORAD), transferred onto nitrocellulose membrane, and the viral protein was visualised using a monoclonal antibody that recognises all IPN viral proteins, and secondary LICOR antibodies.

### Results

### Fine Mapping of IPN Resistance QTL Using Whole Genome Sequence Data

To fine map the IPN resistance QTL, and to identify candidate functional genes and polymorphisms, genomic DNA from salmon fry of known QTL genotype was pooled and whole genome sequencing was performed. These fry were selected from two large IPNV challenge experiments performed on salmon fry in 2007 and 2008. Families where both parents were heterozygous for the QTL were identified (n=11 in 2007, and n=12 in 2008), and from each of those families two homozygous resistant (RR) fish and two homozygous susceptible (SS) fish (total n=22 in 2007, and total n=24 in 2008) were selected for pooling of genomic DNA at equimolar concentrations and sequencing (2× pools of RR fish and 2× pools of SS fish; sequence reads available at NCBI Short Read Archive PRJNA614520) Following alignment of sequence reads to the Atlantic salmon reference genome (GenBank accession GCA_000233375.4), variants were called and the allele frequency differences between the RR and SS pools were calculated (FIG. 1A). The QTL region of chromosome 26 contained the vast majority of the most significant SNPs, with a notable peak at approximately 15 Mb in an intergenic region upstream of the nedd-8 activating enzyme E1 (nae1) gene (FIGS. 1A, 1B).

To screen for putative functional candidate SNPs and indels within the region of the QTL the predicted consequence of all variants was assessed using the SNPEFF software¹⁷. Two missense mutations were identified within the QTL region, one in the epithelial cadherin locus (cdh1) previously identified by Moen et al.⁶, and one in the nae1 locus, which has not previously been reported (FIG. 1B).

To further assess the association between selected high priority SNPs dispersed throughout the QTL region and the putative QTL genotype, a KASP assay was developed for 21 polymorphisms which were subsequently genotyped in individual samples of RR and SS genotypes used in the pooled sequencing experiment. There was no single SNP or indel that showed a perfect concordance with the putative underlying QTL genotype, which is in agreement with Moen et al⁴. However, there were two SNPs in the intergenic region at ˜15 Mb which showed a pattern where all genotyped SS fish across two yeargroups of the breeding population were homozygous for one allele, RR fish were either homozygous for the alternative allele or heterozygous (FIG. 1C), and heterozygous parents were either heterozygous at the SNPs or fixed for the susceptibility-associated SNP allele (data not shown). This pattern is consistent with a dominant-acting primary locus at this location, where a single copy of the resistance-associated allele was sufficient to ensure fish were fully resistant (i.e. survived challenge with IPNV), but also suggests the possibility of a secondary locus acting in the QTL region.

### Contrast in Nae1 Gene Expression Between Resistant and Susceptible Salmon Fry

In order to shortlist candidate genes in the QTL region that may be causative for IPN resistance, global gene expression analyses were performed in RR and SS genotyped individuals from families where both parents were heterozygous for the QTL (families B and C in Houston et al⁸). To achieve this, replicate family-specific tanks (n=50 per tank) were immersion-challenged with IPNV as described in Robledo et al⁶, and whole fry were sampled pre-challenge, 24 hours post-challenge, and 7 days post-challenge. Fry were assigned their QTL genotype using the microsatellite marker panel described in Houston et al⁶, and RR and SS homozygous fry were chosen for gene expression analyses. Whole fry were homogenised, pooled in quadruplicate, and total RNA was extracted.

Global gene expression analyses of pooled ‘RR’ and ‘SS’ individuals revealed that nae1 was the most significant differentially expressed gene within the QTL region (FIG. 2), and one of the most significant genome wide during IPNV infection. Interestingly, nae1 expression was consistently higher in QTL-resistant fry than in QTL-susceptible fry at all timepoints, including constitutively higher expression pre-challenge (FIG. 2).

### IPN Virus Replicates in Both Resistant and Susceptible Fish

Viral load in RR, RS and SS IPNV-challenged fry from families B and C was assessed at day 1, day 7, and day 21 post challenge. Viral load was found to be between 1 and 2 log lower in RR and RS individuals compared with SS individuals, but that all genotypes have viral load that indicate productive replication of the virus. This is consistent with previous reports by Reyes-Lopez et al¹¹ and Robledo et al⁶ in both head kidney and whole fry, which showed an appreciable increase in viral load in fry from both fully resistant and susceptible families during an IPNV challenge. These data demonstrate that the mechanism underlying genetic resistance is not prevention of entry of the virus to the cell, nor the complete prevention of viral replication within the cell.

### CRISPR Knockout of Nae1 Markedly Reduces IPNV Replication in Salmon Cells

Nae1 is an enzyme that is responsible for covalently linking ubiquitin-like protein Nedd8 to target proteins, often modifying their function¹⁴. Inhibition of nae1 activity using a small molecular inhibitor (MLN4924) has been shown to have broad-acting anti-viral activity and to inhibit the replication of a multitude of DNA and RNA viruses in vitro, highlighting the importance of the neddylation process during viral infection15. To assess the role of nae1 in IPNV replication in Atlantic salmon cells, two complementary approaches were taken using the Salmon head kidney (SHK-1) cell line; CRISPR-Cas9 knockout (KO) of the nae1 gene, and MLN4924 inhibition of the nae1 protein activity.

First, CRISPR-Cas9 genome editing was used to knock-out the nae1 gene in SHK-1 cells using recombinant Cas9 protein and custom synthesised gRNAs; a method for high specificity editing of target genes in salmonid cell cultures¹⁰. Exon 2 of the Atlantic salmon nae1 locus was targeted and editing efficiency was 93-97% resulting in 82-87% frameshift mutation (depending on the replicate), highlighting that the vast majority of cells in the mixed cell population were successfully edited. Following IPNV challenge at a multiplicity of infection (MOI) of 0.01, IPNV RNA load and productive viral output were assessed by qRT-PCR and TCID50 assays, respectively. The viral load in the nae 1 KO SHK-1 cell cultures at 96 and 120 hpi was 109.6 and 2.7-fold lower (respectively) than mock-challenged control SHK-1 cells (FIG. 3A, p<0.001 and 0.05). In addition, the infectivity of viral output in the supernatants at 120 hpi was 7.8-fold lower in nae1KO cells (FIG. 3B, p<0.01).

Second, the MLN4924 small molecule inhibitor of nae1 was used in the Atlantic salmon SHK-1 cell line to inhibit nae1 protein function. Cells were treated with 100 nM MLN4924 dissolved in DMSO, or DMSO only as a negative control, for 24 hours prior to infection with IPNV and measurements of viral load and output were taken as described above. Despite little difference in IPNV RNA copy number during the course of infection, there was a substantial (13 to 73-fold) decrease in viral output as measured at 120 hpi in SHK-1 cells (FIG. 3C). In order to confirm this decrease in viral output, western blot against viral proteins was performed on virus purified from SHK-1 cells treated with MLN4924 or DMSO at 120 h (FIG. 3D). There was a notable decrease in the abundance of IPNV viral proteins in cells treated with MLN4924, which demonstrated that inhibition of nae1 activity results in a decrease in viral output. There was no associated decrease in cell viability with the MLN4924 treatment compared to DMSO-treated controls.

Cdh1 is not required for IPNV infection and replication in salmon cells

The IPN resistance QTL was independently reported by Moen et al³ and subsequently the resistance phenotype was partially attributed to a missense variant in the cdh1-1 gene, which encodes a cell surface receptor⁴. This gene was posited to encode a protein, which is required for entry of IPNV into cells. To test this hypothesis and assess the putative role of cdh1 in IPNV infection, cdh1 KO SHK-1 cells were generated using CRISPR-Cas9 genome editing using the method described above. Using a guide RNA that targets exon 2 of cdh1-1, an editing efficiency of 90-94% was observed, resulting in 90-93% frameshift mutation rate (depending on the replicate) in the SHK-1 cells. If cdh1 was critical for the entry and replication of IPNV, viral entry is likely to be prevented in knockout cells, and a marked reduction in viral load in the edited cell culture would be expected. However, while there was a minor (2.1-fold) decrease in viral load measured by qPCR compared to controls at 96 hpi, there was a small increase of 1.3-fold compared to controls at 120 hpi (FIG. 3E). Furthermore, there was no difference in productive viral output between cdh1 KO and control cells as analysed by TCID50 assays (FIG. 3F). This indicates that cdh1 is not essential for the entry of IPNV into salmon cells, nor for successful IPNV replication and productive viral output in these cell lines. To further assess the role of cdh1 in IPN resistance, specific antibodies against the extracellular domain of cdh1 (as used in Moen et a/4) were used to assess whether they block IPNV infection and replication in salmon cells. Despite effective and striking neutralisation of IPNV infection with a specific antibody against IPNV viral protein VP2, there was no indication of an impact of the anti-cdh1 antibody on IPNV replication in the SHK-1.

### Materials and Methods (for Rainbow Trout Experiments

### Cell Culture

The cell line used in this study is the rainbow-trout gonad (RTG-2), which is an immortalized cell line derived from rainbow trout (Oncorhynchus mykiss), obtained from ECACC (product 90102529). The RTG-2 cells were maintained in Leibovitz's-15 (L1518, Sigma-Aldrich, St. Louis, USA) supplemented with 10% fetal bovine serum (FBS) and 100 units/mL penicillin and 100 μg/mL streptomycin solution (Gibco, Waltham, USA). L-15 medium containing 2% FBS (Gibco, Waltham, USA) was used for the TCID50 assay used for virus titration and for the viral challenges.

### Optimisation of Electroporation Settings and Cas9 RNP Genome Editing in RTG-2 Cells

The Rainbow trout Gonad (RTG-2) cell line was tested to develop efficient genome editing methods in vitro using Cas9 ribonucleoprotein (RNP) electroporation and the result has been published previously.¹⁰ Briefly, electroporation settings were optimized by testing several combinations of voltage, pulse duration and number of pulses, as well as two different electroporation buffers. The transfection rates of each different setting combination were measured by flow cytometry. 1400 V, 20 ms, 1 pulse and use of Opti-MEM buffer (Gibco, Waltham, USA) resulted in 99.9-100% transfection rate. Cas9 RNP was transfected in the cells using this electroporation setting and showed 93.5-94.5% genome editing efficiency in a test gene, s/c45a2.

gRNA Design and Formation of Ribonucleic Protein Complex

Guide RNAs were designed to target the coding region of nae1 on rainbow trout chromosome 6 and 26, the homologous regions of the major effect IPNV resistance QTL in Atlantic salmon. Three gRNAs were designed, two targeting nae1 on chromosome 6 and the other targeting both chromosomes 6 and 26. The guide RNAs were chosen based on predicted high cutting efficiency with low number of potential off-targets using CRISPOR (http://crispor.tefor.net/) and Benchling (https://benchling.com/).

### Sequencing

Genomic DNA was extracted from the edited cells 7 and 24dpe using Dynabeads® DNA DIRECT™ Universal kit (Thermo Fisher Scientific, Waltham, USA) and was processed according to the manufacturer's protocol. The DNA samples were amplified in 50 uL PCR reactions, carried out using Q5® Hot Start High-Fidelity 2× Master Mix (New England Biolabs, Ipswich, USA) and 1-3 uL of the extracted gDNA for 35 cycles amplification at optimal annealing temperature (Table 4). PCR samples were then purified using the AMPure XP magnetic beads kit (Agencourt, Beverly, USA) and the DynaMag™-96 Side Magnet plate (Thermo Fisher Scientific, Waltham, USA). Samples sent for Sanger sequencing contained 5 uL of 2 ng/uL (for DNA size up to 300 bp) or 12 ng/uL (for DNA size 300-1000 bp) of purified PCR product, 2.5 uL of 10 uM forward or reverse primer and NFW up to 10 uL. The samples were sent to GATC/Eurofins (Germany) and the sequencing data were received in.abi file format. The results were analyzed and compared to the control (non-edited) corresponding sequence using the Inference of CRISPR Edits (ICE, Synthego Inc).

### IPNV Challenge Model and Methods of Assessing Viral Output

After electroporation, nae1 KO RTG-2 cells and wild-type RTG-2 control cells were seeded in 6 replicate wells (24-well plates). Subsequently, viral challenge was conducted by inoculating each cell population with 10⁻⁵ TCID50/mL or with 10⁻⁵ IPN virus stock dilution (prepared in 2% FBS media with P/S) (1 mL/24-well) and incubation of the inoculated cells at 15° C. for the duration of the experiment. 2 hours post-inoculation, the inoculum was removed and fresh 2% FBS Media (with P/S) was added to the wells (1 mL/24-well). Supernatant was collected and RNA was extracted from the infected cells at two different time points post-inoculation with the virus; 48 and 72 hours. Supernatants and RNA were labelled and stored at −80°° C. until use.

Viral output was measured as viral loads in cells and infectivity in the supernatants. RNA extraction was conducted using the Direct-zol™MRNA Microprep kit (Zymo Research, Irvine, USA), according to the manufacturer's protocol. The viral loads in the cells were assessed by RT-qPCR, using Luna® Universal One-Step RT-qPCR Kit (New England Biolabs, Ipswich, USA). For the detection and relative quantification of the viral RNA present in the cells, the studied gene was the VP2 gene of IPN virus. actb was found to be the most suitable reference gene and was subsequently utilized to normalize the qPCR data for the VP2 gene. The primers for these two genes are included in Table 3.

TABLE 3
Primer sequences for the amplification and
detection of VP2 and actb genes.

| Target | |
|---|---|
| gene | Primers (5′-3′) |
| Actb | Fw: |
| | CTGACGGAGCGCGGTTACAG |
| | Rv: |
| | AAGGAGGGCTGGAAGAGGGC |
| VP2 | Fw: |
| | GACCAAGTTCGACTTCCAGC |
| | Rv: |
| | ATCGGCTTGGTGATGTTCTC |

### MLN4924 Cytotoxicity Test, Treatment and Subsequent IPNV Inoculation

Initially, four different concentrations of MLN4924 0 (DMSO-only), 0.1, 1 and 5 uM, were tested in fresh RTG-2 cells to investigate if they exerted toxicity in wild type RTG-2 cells. A cell viability assay was used to assess any present cytopathic effect at four different time points: 24, 48, 72 and 96 hours post treatment with the four different concentrations of the inhibitor. The results showed that none of the doses were cytotoxic, therefore all four of those were later tested followed by inoculation with the virus, described below.

Wild type RTG-2 cells were seeded in 4× quadruplicates in 24-well plates and were allowed to settle for 24 hours. The four quadruplicates were then treated with DMSO-only, 0.1, 1 and 5 uM of MLN4924, respectively. 24 hours after the MLN4924 treatment, the inhibitor inoculum was removed and IPNV in a dose of 10-5 TCID50/mL was inoculated in the cells, along with the four doses of MLN4924. Supernatants were collected at 48 and 72 hpi, and were stored at −80° C. until use.

### Supernatant Infectivity Assay of MLN4924 and IPNV-treated Cells

TCID50 (Median Tissue Culture Infectious Dose) assay was used to assess the titer of the viral supernatants, harvested from the MLN4924 and IPNV-treated cells at 72 hpi. Firstly, 20,000 cells/well were seeded in wells of a 96-well plate (in four rows of 10 wells for each supernatant, for 4 replicates) and were allowed to settle and grow overnight. The following day, the media was removed with a multichannel pipette and replaced with serial 10-fold dilutions (starting from the neat original viral supernatants up to 10⁻⁷ dilution). The latter were prepared in 2% FBS media (with penicillin streptomycin). The seeded cells were inoculated with these serial dilutions (four replicates per virus dilution, 100 uL/96-well). The last two wells of each row were used as controls, meaning that no virus was added there, only media. The 96-well plate was incubated at 15° C. for the duration of the experiment. The Cytopathic Effect (CPE) of the viral infection was assessed using CellTiter-Glo® Luminescent Cell Viability Assay (Promega, Madison, USA), at 30 hpi. The 50% endpoint titer was further calculated using the Reed and Muench method¹⁶.

### Guide RNAs and PCR Primers

TABLE 4
Guide RNA sequences and primers used for amplification and
sequencing of target genomic regions of the nae1 and s/c45a2 genes,
using Cas9 nuclease.

| gRNA1_nae1_ch6 (1) | GGAGCGTTCACTATTGTAGA | |
|---|---|---|
| | Primer sequence | Tₐ (°C) |
| Fw (ch6) | GCAACATGCCGGTGTTATGA | 63.3 |
| Rv (ch6) | AGCATAATCGTTTACCCTTCCT | |
| Fw (ch26) | TCCACTGCATCCGGGACTGA | 60.7 |
| Rv (ch26) | CGACGTCTTCCCCGGAGACT | |
| gRNA1_nae1_ch6 (2) | TGATCAATTCCACAGCATCT | |
| | | Tₐ (° C.) |
| Fw (ch6) | GCAGCTAGTTAACGTAGTTATGC | 63.3 |
| Rv (ch6) | GAAGAGAGCAACTAGCTTAGG | |
| Fw (ch26) | AACAAGGCATTGGAGATTGG | 59 |
| Rv (ch26) | GAAGAGAGCAACTAGCTTAGG | |
| gRNA3_nae1_ch6&ch26 | TGATGTATCTGGCAACTTTG | |
| | Primer sequence | Tₐ (° C.) |
| Fw (ch6) | AGCAACAACAGCATAGGAAGG | 59 |
| Rv (ch6) | CCCCAAAACTTTTGAACAGTGG | |
| Fw (ch26) | CTAAGCTAGTTGCTCTCTTCATCAGG | 63.3 |
| Rv (ch26) | CACTCAGCAATCAATACAATCAACG | |
| gRNA_slc45a2_ch5 | AGCCCCTTCAGACCGATGTA | |
| | Primer sequence | Tₐ (° C.) |
| Fw | ACCGGAACACAGCAGAAGGGT | 67 |
| Rv | ACAGGTGGTGGATGAGGTTCGCA | |

### Rainbow Trout Results

The purpose of these experiments was to test whether perturbation of the nae1 gene has an impact on IPNV infection in rainbow trout cells. This involved both chemical inhibition of the nae1 protein and CRISPR/Cas9 knockout in immortalized cell lines, followed by challenge with IPNV and assessment of productive viral replication. The methods used followed the general protocols used for Atlantic salmon and are given in detail below.

Viral challenge of nae1 KO cells and viral load quantification

### Knockout of IPNV Resistance Candidate Gene Nae1

Examination of the conservation of the order of the genes showed that the region homologous to the IPNV QTL region in Atlantic salmon (salmon chromosome 26) containing the nae 1 gene is located on chromosome 6 in the rainbow trout genome. However, due to the salmonid whole genome duplication, additional copies of these genes are encountered in chromosome 11 in the Atlantic salmon genome. In rainbow trout, these additional copies are found in chromosome 26. These copies show a high degree of similarity with the genes in the QTL region and therefore, anticipating potential functional redundancy, the copies in chromosome 26 were also targeted.

Guide RNAs were designed to target nae1 gene (GCA_0023375.4 from the NCBI database) on (i) only chromosome 6 (and not simultaneously chromosome 26) and (ii) chromosomes 6 and 26 simultaneously (total of 3 guide RNAs-2 gRNAs targeting nae1 on chromosome 6 and one targeting nae1 on chromosomes 6&26 simultaneously). Primers annealing independently to each of the two regions were designed to assess the editing efficiency in each region specifically (through PCR amplification and Sanger sequencing). Additionally, slc45a2 gene was used as a test gene during the electroporation optimisation but also in the challenges, where it served as an indicator of how the knockout of a ‘random’ gene, most likely not involved in IPNV resistance (knockout of this gene causes albinism in vivo), could affect the susceptibility of the cells to the virus (Table 4).

The editing efficiency (percentage of edited cells) in the target region was estimated using Sanger sequencing of PCR products, as described above. The editing efficiency (percentage of edited cells) of the four designed gRNAs had been tested previously and is generally high.

### Viral Challenge of Edited KO Cells

Viral challenge was conducted by inoculating 17×10⁴ cells/well (24-well) with 10⁻⁵ TCID50/mL of the initial virus suspension and incubation of the inoculated cells at 15° C. for the duration of the experiment. In this viral challenge, the edited cell populations were sourced from a common electroporation, the results of which are presented in Table 5, and cells were challenged with the virus 24 days post electroporation (dpe) while being on passage number 31. The wild type electroporated without Cas9/RNP cell population was used as a control for the normalization of the qPCR data of the other KO cell populations.

TABLE 5
Editing percentages achieved in the cell
populations used in the viral challenge.

| gRNA | KO SCORE |
|---|---|
| gRNA slc45a2 | 33% |
| gRNA nae1 ch6 (1) | 87% |
| gRNA nae1 ch6 (2) | 73% |
| gRNA nae1 ch6&ch26 (1) | 67% (ch6)-49% (ch26) |
| gRNA nae1 ch6&ch26 (2) | 74% (ch6)-62% (ch26) |

After their inoculation with the virus, cells were harvested at 2 different time points: 48 and 72 hpi and RNA was extracted from the all the cells in each well. Subsequently, RT-qPCR analysis of the viral load on the RNA samples of each time point provided C_{q} values Reactions with more than one peak in the RT-qPCR melting curve, indicating possible contamination, were excluded from further analysis, and the remaining samples were further analyzed and normalized against the control cell population. The 2^{-(ΔΔCq)} values showing differences in viral load between the different edited cell populations are shown in FIG. 4.

The most profound difference in the viral load present in the cells is the one projected by nae1 KO on both chromosomes 6&26 (bar shaded in blue and brown). This trend appears downward when compared to the control (shaded in green), with nae1 knocked out in both chromosomes exhibiting statistically significant difference (*p≤0.05) at 72 hours post inoculation (hpi).

### Inhibition of NAE1 Using the Specific Inhibitor MLN4924

### Supernatant Infectivity Assay of the 72 hpi Supernatants

MLN4924 inhibitor was used to inhibit nae1 activity. Four supernatants of each dose group collected from the MLN4924 and IPNV-treated cells at 72 hpi were tested in two independent TCID50 infectivity assays followed by a cell viability assay at 30 hpi and use of the Reed and Muench method to analyze the cytopathic effect present in the wells. The results of the two independent TCID50 assays are presented in FIG. 5. Data for the 0.1 μM MLN4924 dose are not shown, since those exhibited an effect similar to the DMSO-only cell group.

There appears to be a marked drop in the productive IPNV viral output in MLN4924 treated cells, for the 1 and 5 μM doses of the inhibitor, when compared to the DMSO-only treated cells.

### Discussion

It has been well documented that resistance to IPN in Atlantic salmon has a major genetic component, and the majority of variation in mortality observed between resistant and susceptible fish can be explained by a major QTL on chromosome 262.3. However, the causative mutations and the underlying molecular biology of the resistance phenotype were not well understood. In the current study, whole genome sequencing of salmon fry with known QTL genotypes was used to fine map the most significant SNPs and indels to a region upstream of the nae1 gene. Global gene expression profiling highlighted differentially expressed between susceptible and resistant fish prior to and during IPNV infection, and this revealed that nae1 is one of the most significant differentially expressed genes genome-wide, and the most significant in the QTL region. Finally, the perturbation of the two primary candidate genes within the IPN QTL was tested using salmon cell line models; cdh1 (as proposed by Moen et al.⁵), and nae1 based on evidence in the current study.

The whole genome resequencing revealed two missense coding mutations, one in nae1 and one in cdh1. The genotyping results highlighted that no single SNP or indel was fully concordant with the QTL genotype, but a cluster of SNPs in this region were all found to be homozygous for one allele in susceptible fish, and either heterozygous or homozygous for the alternative allele in resistant fish. These findings may be consistent with local epistasis, with a dominant acting primary resistance locus, or with a further (unidentified) secondary locus or loci in the region associated with the QTL effect in fish fixed for the susceptibility allele at this primary locus. These findings are generally consistent with results using a similar approach by Moen et al., although the location of the most significant SNPs differs⁴. The results of the gene expression comparison showed nae1 to be one of the most significant differentially expressed genes between susceptible and resistant fish, both in the pre-challenge fry and at all measured timepoints post challenge. This highlights the possibility that the intergenic region located ˜15 Mb on chromosome 26 contains regulatory elements for nae1 expression, or that the nae1 missense SNP alters the expression of the gene (either directly or indirectly). Interestingly although higher expression was associated with resistance in these IPNV challenged fry, the downstream functional experiments suggest that lack of functional nae1 activity is linked to reduction in productive viral replication.

Nae1 is an enzyme responsible for the covalent attachment of nedd8, an ubiquitin like modifier, to substrate proteins. Neddylation primarily functions to activate the cullin-RING ligases that in turn regulate the degradation of specific substrates via ubiquitination¹⁴. In the current study, for IPNV in Atlantic salmon, nae1 knockout or chemical inhibition results in significant decrease in productive viral replication (FIG. 3). Neddylation plays a significant role in the stimulation of the host type 1 interferon response to viral infections, and many viruses attempt to evade the host immune response by targeting type I interferon signalling¹⁷. Both IRF3 and IRF7 were amongst the most significantly differentially expressed genes between RR and SS fry following IPNV challenge in the current study, showing higher expression in susceptible fish, and highlighting their importance in IPNV host response. It is conceivable that the genetic variants identified in the nae1 regulatory or coding regions lead to an alteration of neddylation function in resistant fish. As a consequence this would result in a modified type 1 interferon response, potentially due to changes in IRF3 and IRF7 signalling, which may protect IPNV infected fish from the damaging cytokine storm which is postulated to be a major cause of IPN morbidity¹⁸.

A SNP within the E-cadherin gene (cdh1) has previously been proposed as a functional variant which leads to IPN resistance in Atlantic salmon⁴. The proposed mechanism was that Cdh1 acts as the receptor for IPNV to enter cells via clathrin-mediated endocytosis, and the causative SNP blocks IPNV binding and/or entry. While IPNV has been shown to bind to cdh1⁴, it is unlikely that this is the sole route of entry during infection. Reyes-Lopez et al.¹¹, Robledo et al.⁶, and the findings presented herein show that resistant fish do become infected with IPNV and with viral load levels that can only be explained by successful replication in cells of fully (homozygous) resistant salmon fry. It has also recently been demonstrated that macropinocytosis is the primary route for IPNV entry into SHK-1 and CHSE-214 cells, a process that is likely to be non-discriminatory and not reliant on a specific receptor19. To assess this further in the current study, CRISPR-Cas9 editing was used to knockout cdh 1 with high efficiency in salmon cell culture. When these KO cells were challenged with IPNV there was limited evidence for an impact on viral load, and no evidence for an impact on productive viral output when compared with wild-type cells, indicating that cdh1 is not essential for viral entry or replication in these cells (FIG. 3E). Furthermore, using an antibody against e-cadherin, it was not possible to block IPNV entry or inhibit replication in Atlantic salmon cells in culture in the current. While it is plausible that cdh1 plays a role in IPN resistance at the site of infection in vivo (e.g. in gut epithelia), the results presented herein do not support a major role for cdh1 in IPN resistance. In contrast, the genetic mapping, gene expression, and functional virology experiments all provide evidence for a major role of nae1 in underlying the major IPN resistance QTL.

The IPN QTL has become a well-known exemplar of the application of molecular genetics to tackle a major infectious disease problem in farmed animals1. Application of marker-assisted selection for the resistance allele has reduced incidence of disease outbreaks close to zero in all the major salmon-producing countries1. While identification of the underlying causative gene and mechanisms is of limited practical utility to disease control in salmon aquaculture, IPN is also a serious pathogen of other salmonid species, including rainbow trout. Prior to the present disclosure and unlike salmon, there is no evidence for an equivalent major QTL affecting IPN resistance segregating in commercial rainbow trout populations. The results shown herein, support a role for Nae1 being a significant resistance allele.

### Conclusions

Fine mapping of the major IPNV resistance QTL using whole genome sequencing combined with differential expression between homozygous resistant and homozygous susceptible fish both pointed to nae1 as a strong candidate causative gene. Functional assessment of CRISPR-Cas9 knockout of nae1, and specific inhibition of the nae1 protein activity in IPNV-challenged salmon cells revealed a marked decrease in productive viral output. A previously identified candidate gene cdh1 has been suggested to be the cellular receptor for IPNV, with resistance due to prevention of viral entry to cells. However, in the current study, prevention of IPNV binding to cdh1 either via CRISPR-Cas9 knockout of cdh1 or binding of a cdh1 antibody did not influence productive IPNV replication. Further work looking at nae1 in Rainbow trout has shown that inhibition of NAE1 leads to a significant reduction in viral output in a controlled infection. Taken in combination, these results show that nae1 is the likely causative gene underlying the major IPN QTL, which highlights the extensive role of neddylation in immune response to a broad range of viral infections.

### REFERENCES

1. Houston, R. D. et al. Harnessing genomics to fast-track genetic improvement in aquaculture. Nature Reviews Genetics 1-21 (2020) doi: 10.1038/s41576-020-0227-y.

2. Houston, R. D. et al. Major quantitative trait loci affect resistance to infectious pancreatic necrosis in Atlantic salmon (Salmo salar). Genetics 178, 1109-15 (2008).

3. Moen, T., Baranski, M., Sonesson, A. K. & Kjøglum, S. Confirmation and fine-mapping of a major QTL for resistance to infectious pancreatic necrosis in Atlantic salmon (Salmo salar): population-level associations between markers and trait. BMC Genomics 10, 368 (2009).

4. Moen, T. et al. Epithelial cadherin determines resistance to infectious pancreatic necrosis virus in Atlantic salmon. Genetics 200, 1313-26 (2015).

5. Roberts, R. J. & Pearson, M. D. Infectious pancreatic necrosis in Atlantic salmon, Salmo salar L. J. Fish Dis. 28, 383-390 (2005).

6. Robledo, D. et al. Gene expression comparison of resistant and susceptible Atlantic salmon fry challenged with Infectious Pancreatic Necrosis virus reveals a marked contrast in immune response. BMC Genomics 17, 279 (2016).

7. Gratacap, R. L., Wargelius, A., Edvardsen, R. B. & Houston, R. D. Potential of genome editing to improve aquaculture breeding and production. Trends Genet. 35, 672-684 (2019).

8. Houston, R. D. et al. The susceptibility of Atlantic salmon fry to freshwater infectious pancreatic necrosis is largely explained by a major QTL. Heredity (Edinb). 105, 318-327 (2010).

9. Taggart, J. B. et al. A description of the origins, design and performance of the TRAITS-SGP Atlantic salmon Salmo salar L. cDNA microarray. J. Fish Biol. 72, 2071-2094 (2008).

10. Gratacap, R. L., Jin, Y. H., Mantsopoulou, M. & Houston, R. D. Efficient Genome Editing in Multiple Salmonid Cell Lines Using Ribonucleoprotein Complexes. Mar. Biotechnol. 1-8 (2020) doi: 10.1007/s10126-020-09995-y.

11. Reyes-López, F. E. et al. Differential immune gene expression profiles in susceptible and resistant full-sibling families of Atlantic salmon (Salmo salar) challenged with infectious pancreatic necrosis virus (IPNV). Dev. Comp. Immunol. 53, 210-221 (2015).

12. REED, L. J. & MUENCH, H. A SIMPLE METHOD OF ESTIMATING FIFTY PER CENT ENDPOINTS12. Am. J. Epidemiol. 27, 493-497 (1938).

13. Cingolani, P. et al. A program for annotating and predicting the effects of single nucleotide polymorphisms, SnpEff: SNPs in the genome of Drosophila melanogaster strain w1118; iso-2; iso-3. Fly (Austin). 6, 80-92 (2012).

14. Enchev, R. I., Schulman, B. A. & Peter, M. Protein neddylation: Beyond cullin-RING ligases. Nature Reviews Molecular Cell Biology vol. 16 30-44 (2015).

15. Le-Trilling, V. T. K. et al. Broad and potent antiviral activity of the NAE inhibitor MLN4924. Sci. Rep. 6, 1-14 (2016).

16. Ramakrishnan, M. A. Determination of 50% endpoint titer using a simple formula. World J. Virol. 5, 85-86 (2016).

17. Han, K. & Zhang, J. Roles of neddylation against viral infections. Cellular and Molecular Immunology vol. 15 292-294 (2018).

18. Teijaro, J. R. Cytokine storms in infectious diseases. Seminars in Immunopathologyvol. 39 501-503 (2017).

19. Levican, J. et al. Infectious pancreatic necrosis virus enters CHSE-214 cells via macropinocytosis. Sci. Rep. 7, 1-12 (2017).

20. Van der Auwera, G. A. et al. From fastQ data to high-confidence variant calls: The genome analysis toolkit best practices pipeline. Curr. Protoc. Bioinforma. 43, (2013).

1. A genetically modified salmonid fish, wherein the fish has been genetically modified such that its genome comprises, consists essentially of, or consists of a mutant nae1 gene or allele.

2. The genetically modified salmonid fish according to claim 1, wherein the mutant nae1 gene or allele comprises a substitution, deletion, inversion, addition or multiplication of one or more nucleotides, within the nae1 gene sequence, or in a regulatory region adjacent to the nae1 gene, which is associated with expression of nae1.

3. The genetically modified salmonid fish according to claim 1, wherein the mutant nae1 gene or allele results in a reduction in NAE1 protein and/or activity levels.

4. The genetically modified salmonid fish according to claim 1, which displays increased resistance to virus infection as compared to a corresponding wild-type salmonid fish which does not comprise the mutant nae1 gene or allele.

5. A method of breeding a population of salmonid fish displaying increased virus infection resistance, the method comprising providing at least one male genetically modified salmonid fish and at least one female genetically modified salmonid fish according to claim 1 and breeding the population of salmonid fish displaying increased virus infection resistance from said at least one male genetically modified salmonid fish and said at least one female genetically modified salmonid fish.

6. (canceled)

7. (canceled)

8. A method of identifying whether or not a salmonid fish may display increased resistance to infection by a virus, the method comprising detecting an nucleotide alteration, expression or activity level of a nae1 gene and/or protein in the salmonid fish and determining whether or not the salmonid fish is resistant, or likely to display increased resistance to infection, or likely to have offspring which display increased resistance to infection by the virus, based on the nucleotide alteration, expression or activity level detected.

9. The method according to claim 8 wherein a reduced level of expression or activity of the nae1 gene and/or protein, is associated with fish which are likely to display an increased resistance to infection by a virus.

10. The method according to claim 9 further comprising selecting a fish which displays a reduced level of expression or activity of the nae1 gene and/or protein for use a broodstock.

11-13. (canceled)

14. A method of treating or preventing virus infection in salmonid fish, the method comprising administering a NAE1 protein inhibitor to the salmonid fish or a population of salmonid fish.

15. (canceled)

16. The method according to claim 14, wherein the NAE1 inhibitor is MLN4924 (pevonedistat).

17. The method of claim 14, wherein the virus is a birnavirus.

18. The method according to claim, wherein the virus is infectious pancreatic necrosis virus (IPNV).

19. The method according to claim 14, wherein the salmonid fish is selected from the group consisting of salmon, Atlantic, Sockeye, Steelhead, Coho, Chinook salmon, trout, rainbow trout, brown trout, chars, freshwater whitefishes, and graylings.

20. (canceled)


SEQUENCE LISTING (32 sequences)

SEQ ID NO: 1 - RNA, 20 nt, synthetic construct
  source 1..20: mol_type = other RNA
tgatcaattccacagcatct

SEQ ID NO: 2 - DNA, 22 bp, synthetic construct
  source 1..22: mol_type = other DNA
atgccagccaaccagcatgctt

SEQ ID NO: 3 - DNA, 22 bp, synthetic construct
  source 1..22: mol_type = other DNA
ttccgacgtcttccccggagac

SEQ ID NO: 4 - RNA, 20 nt, synthetic construct
  source 1..20: mol_type = other RNA
tcggagtcaacatgtctacc

SEQ ID NO: 5 - DNA, 22 bp, synthetic construct
  source 1..22: mol_type = other DNA
ccacatttcgcaatcgggtgaa

SEQ ID NO: 6 - DNA, 22 bp, synthetic construct
  source 1..22: mol_type = other DNA
atttcccagtcggagctcgttt

SEQ ID NO: 7 - DNA, 19 bp, synthetic construct
  source 1..19: mol_type = other DNA
ggctggttcaagggatgga

SEQ ID NO: 8 - DNA, 19 bp, synthetic construct
  source 1..19: mol_type = other DNA
cagagtcacaccattggcg

SEQ ID NO: 9 - DNA, 20 bp, synthetic construct
  source 1..20: mol_type = other DNA
gaccaagttcgacttccagc

SEQ ID NO: 10 - DNA, 20 bp, synthetic construct
  source 1..20: mol_type = other DNA
atcggcttggtgatgttctc

SEQ ID NO: 11 - DNA, 20 bp, synthetic construct
  source 1..20: mol_type = other DNA
ctgacggagcgcggttacag

SEQ ID NO: 12 - DNA, 20 bp, synthetic construct
  source 1..20: mol_type = other DNA
aaggagggctggaagagggc

SEQ ID NO: 13 - DNA, 20 bp, synthetic construct
  source 1..20: mol_type = other DNA
gaccaagttcgacttccagc

SEQ ID NO: 14 - DNA, 20 bp, synthetic construct
  source 1..20: mol_type = other DNA
atcggcttggtgatgttctc

SEQ ID NO: 15 - RNA, 20 nt, synthetic construct
  source 1..20: mol_type = other RNA
ggagcgttcactattgtaga

SEQ ID NO: 16 - DNA, 20 bp, synthetic construct
  source 1..20: mol_type = other DNA
gcaacatgccggtgttatga

SEQ ID NO: 17 - DNA, 22 bp, synthetic construct
  source 1..22: mol_type = other DNA
agcataatcgtttacccttcct

SEQ ID NO: 18 - DNA, 20 bp, synthetic construct
  source 1..20: mol_type = other DNA
tccactgcatccgggactga

SEQ ID NO: 19 - DNA, 20 bp, synthetic construct
  source 1..20: mol_type = other DNA
cgacgtcttccccggagact

SEQ ID NO: 20 - RNA, 20 nt, synthetic construct
  source 1..20: mol_type = other RNA
tgatcaattccacagcatct

SEQ ID NO: 21 - DNA, 23 bp, synthetic construct
  source 1..23: mol_type = other DNA
gcagctagttaacgtagttatgc

SEQ ID NO: 22 - DNA, 21 bp, synthetic construct
  source 1..21: mol_type = other DNA
gaagagagcaactagcttagg

SEQ ID NO: 23 - DNA, 20 bp, synthetic construct
  source 1..20: mol_type = other DNA
aacaaggcattggagattgg

SEQ ID NO: 24 - DNA, 21 bp, synthetic construct
  source 1..21: mol_type = other DNA
gaagagagcaactagcttagg

SEQ ID NO: 25 - RNA, 20 nt, synthetic construct
  source 1..20: mol_type = other RNA
tgatgtatctggcaactttg

SEQ ID NO: 26 - DNA, 21 bp, synthetic construct
  source 1..21: mol_type = other DNA
agcaacaacagcataggaagg

SEQ ID NO: 27 - DNA, 22 bp, synthetic construct
  source 1..22: mol_type = other DNA
ccccaaaacttttgaacagtgg

SEQ ID NO: 28 - RNA, 26 nt, synthetic construct
  source 1..26: mol_type = other RNA
ctaagctagttgctctcttcatcagg

SEQ ID NO: 29 - DNA, 25 bp, synthetic construct
  source 1..25: mol_type = other DNA
cactcagcaatcaatacaatcaacg

SEQ ID NO: 30 - RNA, 20 nt, synthetic construct
  source 1..20: mol_type = other RNA
agccccttcagaccgatgta

SEQ ID NO: 31 - DNA, 21 bp, synthetic construct
  source 1..21: mol_type = other DNA
accggaacacagcagaagggt

SEQ ID NO: 32 - DNA, 23 bp, synthetic construct
  source 1..23: mol_type = other DNA
acaggtggtggatgaggttcgca